(19) United States
(12) Patent Application Publication
(10) Pub. No.: US 2009/0254016 A1
(43) Pub. Date: Oct. 8, 2009

(54) Method and apparatus for cushioning wrists

(76) Inventor: Max Joseph Wallack, Natick, MA (US)

Correspondence Address: Max Wallack, 9 Penacook Lane, Natick, MA, 01760, US

(21) Appl. No.: 12/099,722

(22) Filed: Apr. 8, 2008

(51) Int. Cl. A61F 5/00

(52) U.S. Cl. 602/21

(57) ABSTRACT

An apparatus is provided for cushioning wrists using sealed air. The invention can alleviate suffering due to carpal tunnel syndrome and the invention may even prevent carpal tunnel syndrome. The invention provides comfortable support by using air filled cushioning element to provide support. The invention positions the sealed air cushioning element beneath the wrist so that the wrist rests on the sealed air cushioning element and the sealed air cushioning element rests on a surface. The invention maintains the position of the sealed air cushioning element to be directly beneath the wrist by placing the sealed air cushioning element in a pocket and affixing the pocket to two straps which strap around the back of the hand.

### FIELD OF THE INVENTION

The invention relates to wrist support, and particularly to systems for alleviating suffering from carpal tunnel syndrome.

### BACKGROUND OF THE INVENTION

Carpal tunnel syndrome is an affliction which causes, pain in people's hands and wrists. Carpal tunnel syndrome is believed to be caused by repetitive motions. Carpal tunnel syndrome affects typists, data entry operators, computer programmers, factory workers, and agricultural workers.

Many people suffer from carpal tunnel syndrome. It is believed that 10% of workers will suffer from carpal tunnel syndrome at some point in their lives. It is estimated that, at any given time, 0.03% of workers are sufferinAgfrom carpal tunnel syndrome.

Carpal tunnel syndrome is also called median neuropathy of the wrist. Carpal tunnel syndrome is a medical condition in which the median nerve is compressed at the wrist, leading to pain, paresthesias, and muscle weakness in the forearm and hand. The median nerve passes through the carpal tunnel, a canal in the wrist that is surrounded by bone on three sides, and a transverse carpal ligament on the fourth. Nine tendons—the flexor tendons of the hand—through this canal.

Many people with carpal tunnel syndrome have gradually increasing symptoms over time. The first symptoms of CTS may appear when sleeping, and typically include numbness and paresthesia (a burning and tingling sensation) in the fingers, especially the thumb, index, and middle fingers. Difficulty gripping and making a fist, dropping objects, and weakness are symptoms of progression.

Most cases of CTS are idiopathic: without a known cause. A common factor in developing carpal tunnel symptoms is increased hand use or activity. While repetitive activities are often blamed for the development of CTS, the correlation is often unclear. The international debate regarding the relationship between CTS and repetitive motion and work is ongoing. The Occupational Safety and Health Administration (OSHA) has adopted rules and regulations regarding cumulative trauma disorders. Occupational risk factors of repetitive tasks, force, posture, and vibration have been cited. However, the American Society for Surgery of the Hand has issued a statement that the current literature does not support a causal relationship between specific work activities and the development of diseases such as CTS.

The relationship between work and CTS is controversial; in many locations workers injured at work are entitled to time off and compensation. Many cases of carpal tunnel syndrome are provoked by repetitive grasping and manipulating activities, and the exposure can be cumulative. Symptoms are commonly exacerbated by forceful and repetitive use of the hand and wrists in industrial occupations. Studies done by the National Institute for Occupational Safety and Health (NIOSH) indicated that job tasks involving highly repetitive manual acts or necessitating wrist bending or other stressful wrist postures were connected with incidents of CTS or related problems.

In particular, common activities that have been identified as contributing to repetitive stress induced carpal tunnel syndrome include typing on a computer keyboard or typewriter. Often people suffering from carpal tunnel syndrome can have multiple contributing factors which are aggravated by vigorous hand activities and repetitive stress trauma to the hand. Proper attention to ergonomic considerations can reduce or eliminate these kinds of exposures.

The National Institute of Neurological Disorders and Stroke which is part of the National Institutes of Health makes the following recommendation:
At the workplace, workers can do on-the-job conditioning, perform stretching exercises, take frequent rest breaks, wear splints to keep wrists, straight, and use correct posture and wrist position. Wearing fingerless gloves can help keep hands warm and flexible. Workstations, tools and tool handles, and tasks can be redesigned to enable the worker's wrist to maintain a natural position during work. Jobs can be rotated among workers. Employers can develop programs in ergonomics, the process of adapting workplace conditions and job demands to the capabilities of workers. However, research has not conclusively shown that these workplace changes prevent the occurrence of carpal tunnel syndrome.

For users of video display terminals, the National Institute for Occupational Safety and Health says that cushioned and broad support services (e.g., chair arm rest or wrist rest) for the upper extremities can help to minimize compression or irritation at the wrist, forearm, or elbow.

In addition to cushioned and broad support services, it is also important that one's body be aligned properly with the keyboard. This is most easily accomplished by bending ones elbows to a 90 degree angle and making sure the keyboard is at the same height as the elbows. Also it is important not to put physical stress on the wrists by hanging the wrist on the edge of a desk, or exposing the wrists to strong vibrations (e.g. manual lawn mowing). Position the computer monitor directly in front of your seat, so the neck is not twisted to either side when viewing the screen.

One recommended treatment for carpal tunnel syndrome involves immobilizing the wrist via stiff braces and splints. Braces and splints can be heavy, and stiff, making them uncomfortable to wear. In addition, healing braces can sometimes exacerbate the cause of wrist pain and misalignment by continuing to prohibit proper functionality of the wrist.

### SUMMARY OF THE INVENTION

The invention is an apparatus that is a cushion that supports user's wrist that uses in an illustrative embodiment sealed air to provide cushioning and positions that cushioning beneath the wrist. The user wears the exemplary apparatus on a hand so that the user's wrist rests on the sealed air cushion and the sealed air cushion rests on the supporting surface. The user can choose to wear one invention on each hand. The apparatus does not limit the user's range of motion and therefore the apparatus may remain on the user's hand even when the user is not resting their wrists on a surface.

The invention is designed to alleviate suffering due to Carpal Tunnel Syndrome by cushioning the user's wrist.

In an illustrative embodiment, the apparatus positions a small sheet of Bubble Wrapr™ under the user's wrist. The Bubble Wrap™ is secured inside a pocket. The pocket is held in place beneath the user's wrist by means of two adjustable straps. The two adjustable straps wrap around the back of the hand, and are affixed around the hand by Velcro™ fasteners.

In an illustrative embodiment, the pocket containing the Bubble Wrap™ sheet can be closed to hold the Bubble Wrap™ in place. The pocket has an opening on one side and a flap which closes over that opening. The flap has a Velcro™ fastener which can hold the flap closed.

The pocket containing the Bubble Wrap™ sheet can be repeatedly opened and closed so that the Bubble Wrap™ sheet can be replaced if necessary.

The invention can use cushioning elements other than Bubble Wrap™. In some embodiments, the cushioning element could alternatively be a plurality of separate plastic coated air bubbles. In some embodiments, the cushioning element could alternatively be a single plastic coated air bubble. In some embodiments, the cushioning element could alternatively be a water-filled packet.

The pocket has a bottom face which contacts the support surface and a top face which contacts the user's palm and wrist. In some embodiments, the top face of the pocket which contacts the user's palm and wrist is made out of soft material so that it feels comfortable to the user. In some embodiments, the bottom face is made out of studier material than the top face because the bottom face contacts possibly rough support surfaces.

### BRIEF DESCRIPTION OF THE DRAWINGS

FIG. 1 is a depiction of an embodiment of the invention

FIGS. 2a and 2b are depictions of an embodiment of the invention affixed onto a user's hand

FIG. 3 is a depiction of an alternate embodiment of the invention, which includes a flap to close the pocket.

FIG. 4 is a depiction of an alternate embodiment of the invention which includes a flap to close the pocket.

FIG. 5 is a diagram of an alternate embodiment of the invention which uses a water filled balloon as the cushioning element.

FIG. 6 is a diagram of an alternate embodiment of the invention which does not include a pocket and uses a water filled balloon as the cushioning element.

FIG. 7 is a diagram of an alternate embodiment of the invention which uses snaps to affix the straps

### DETAILED DESCRIPTION OF THE ILLUSTRATIVE EMBODIMENTS

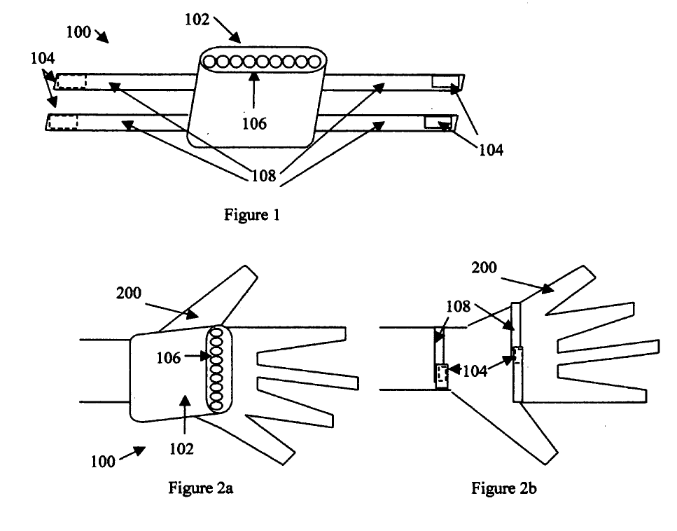

FIG. 1 is a diagram of an embodiment of the invention 100. Sealed air cushioning element 106 is contained within pocket 102. Four strips 108 are attached to pocket 102. Each strip 108 includes a Velcro™ fastening mechanism 104 to fasten it to another strip 108. Two of the Velcro™ fastening mechanisms 104 are shown using dashed lines because the fastening devices are on the opposite side of the straps 108. Other Velcro™ fastener configurations may also be used, such as even longer pieces of Velcro™, multiple placements of Velcro™, or Velcro™ only on one side, for example.

FIGS. 2a and 2b are diagrams of an embodiment of the invention 100 affixed onto a user's hand 200. FIG. 2a is a diagram of an embodiment of the invention 100 affixed onto a user's hand 200 shown from below the hand. FIG. 2b is a diagram of an embodiment of the invention 100 affixed onto a user's hand 200 shown from above the hand. In FIG. 2a, sealed air cushioning element 106 is contained within pocket 102. In FIG. 2b, four strips 108, which are attached to pocket 102, wrap around the top of the hand. The two corresponding pairs of strips 108 are affixed together using a Velcro™ fastening mechanism 104. Two of the Velcro™ fastening mechanisms 104 are shown using dashed lines because the fastening devices are on the opposite side of the straps 108.

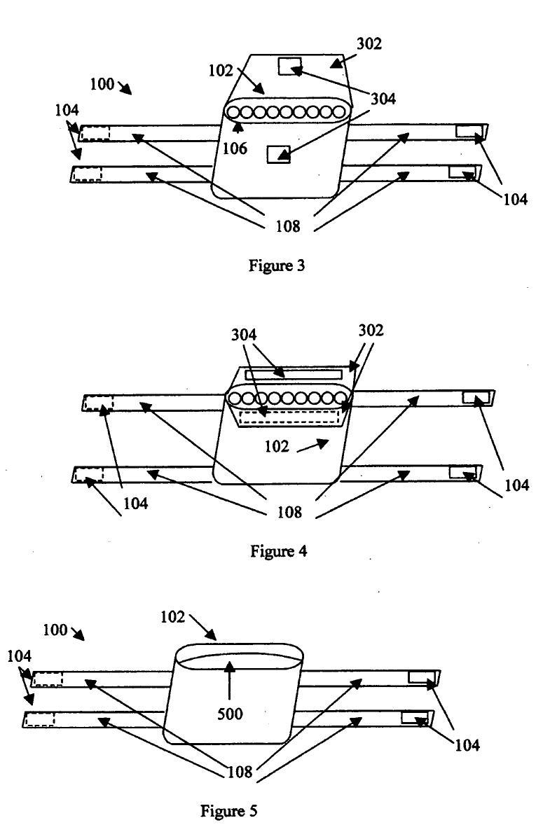

FIG. 3 is a diagram of an alternate embodiment of the invention 100 which includes a flap to close the pocket. Sealed air cushioning element 106 is contained within pocket 102. Flap 302 can close pocket 102 in order to secure cushioning element 106. Velcro™ fastening mechanisms 304 can be used to fasten flap 302 closed. Four strips 108 are attached to pocket 102. Each strip 108 includes a Velcro™ fastening mechanism 104 to fasten it to another strip 108. In one embodiment two of the four strips that fasten together are called component strips. Two of the Velcro™ fastening mechanisms 104 are shown using dashed lines because the fastening devices are on the opposite side of the straps.

FIG. 4 is a depiction of an alternate embodiment of the invention 100 which includes a flap to close the pocket. Sealed air cushioning element 106 is contained within pocket 102. Flap 302 can close pocket 102 in order to secure cushioning element 106. Velcro™ fastening mechanisms 304 can be used to fasten flap 302 closed. Four strips 108 are attached to pocket 102. Note that the four straps 108 are placed at the extreme ends of the pocket 102 in order to maintain the position of pocket 102. Each strip 108 includes a Velcro™ fastening mechanism 104 to fasten it to another strip 108. Two of the Velcro™ fastening mechanisms 104 are shown using dashed lines because the fastening devices are on the opposite side of the straps 108. One of the Velcro™ fastening mechanisms 304 is shown using dashed lines because the fastening devices are on the opposite side of the pocket 302.

FIG. 5 is a diagram of an alternate embodiment of the invention 100 which uses a water filled balloon 500 as the cushioning element. Sealed water cushioning element 500 is contained within pocket 102. Four strips 108 are attached to pocket 102. Each strip 108 includes a Velcro™ fastening mechanism 104 to fasten it to another strip 108. Two of the Velcro™ fastening mechanisms 104 are shown using dashed lines because the fastening devices are on the opposite side of the straps 108.

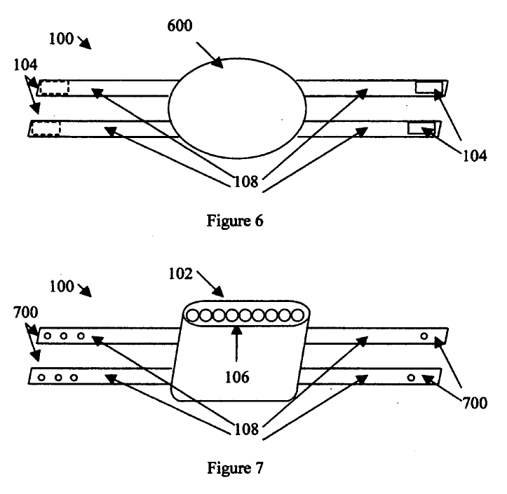

FIG. 6 is a diagram of an alternate embodiment of the invention 100 which does not include a pocket and uses a water filled balloon 600 as the cushioning element. Four strips 108 are attached to sealed water cushioning element 600. Each strip 108 includes a Velcro™ fastening mechanism 104 to fasten it to another strip 108. Two of the Velcro™ fastening mechanisms 104 are shown using dashed lines because the fastening devices are on the opposite side of the straps 108.

FIG. 7 is a diagram of an alternate embodiment of the invention 100 which uses snaps to affix the straps. Sealed air cushioning element 106 is contained within pocket 102. Four strips 108 are attached to pocket 102. Each strip 108 includes a snap fastening mechanism 700 to fasten it to another strip 108. Each snap fastening mechanism 700 consists of a plurality of snaps in order to provide adjustable sizing.

Other elements may provide the cushioning without departing from the scope of the invention, such as gel, for example. Other fastening/pocket configurations may be used without departing from the scope of the invention such as one wide strap or a fabric envelope without a compartment for the cushioning, for example, as long as the cushioning element is held sufficiently in place, the mechanism is within the scope of the invention. Also, other fastening mechanisms may be used as long as the cushioning element is secured to the wrist.

Other pocket or pocketless variations may also be used without departing from the scope of the invention, such as half pockets, for example, as long as the cushioning element is held sufficiently in place to provide the cushioning necessary.

Alternative embodiments that combine one or more of the pocket, flap, fluid/air, and/or fasteners is within the scope of the invention.

What is claimed is:

1. An apparatus for cushioning a user's wrist, the apparatus comprising:
A plurality of filled bubbles; and
At least one support adapted to holding the bubbles substantially below a wrist of a user.

2. The apparatus of claim 1, where the plurality of fluid filled bubbles comprise air filled bubbles.

3. The apparatus of claim 1, where the plurality of fluid filled bubbles comprise regularly spaced.

4. The apparatus of claim 1, where the plurality of fluid filled bubbles comprise affixed together in sheets.

5. The apparatus of claim 4, where the plurality of fluid filled bubbles comprise sheets of Bubble-Wraps™.

6. The apparatus of claim 1, wherein the support further comprises:
A pocked adapted to support the plurality of filled bubbles; and
At least one fastener adapted to hold the pocket substantially below the user's wrist.

7. The apparatus of claim 6, wherein the pocket has an open state and closed state, the closed state adapted to securely contain the plurality of fluid filled bubbles in the pocket and wherein the pocket is further adapted to position the plurality of fluid filled bubbles.

8. The apparatus of claim 7, wherein the pocket can r move between the open state and closed state thereby allowing replacement of the plurality of fluid filled bubbles.

9. The apparatus of claim 7, wherein the pocket further comprises a a flap that can be closed.

10. The apparatus of claim 8, wherein the pocket includes a flap that can be opened and closed.

11. The apparatus of claim 9, wherein the flap is further adapted to be fastened closed.

12. The apparatus of claim 10, wherein the flap is further adapted to be fastened closed and be repeatedly opened and closed.

13. The apparatus of claim 12, wherein the flap comprises a hook and loop fastener to fasten closed the flap, the hook and loop fastener are adapted to repeatable open and close

14. The apparatus of claim 1, wherein the support is further adapted to be adjustable.

15. The apparatus of claim 1, where the support is further adapted to wraps around a back of a hand of the user.

16. The apparatus of claim 15, wherein the support further comprises two fasteners, the two fasteners being adapted to wrap around the back of a hand of the user.

17. The apparatus of claim 16, wherein the at least one of the two fasteners further comprises two component fasteners, one of the component fasteners being adapted to fasten below the wrist of the user and the other of the two component fasteners being adapted to fasten above the wrist of the user.

18. The apparatus of claim 1, wherein the at least one fastener is adapted to be repeatedly opened and closed.

19. The apparatus of claim 18, wherein the at least one fastener further comprises a hook and loop fastener adapted to be repeatedly opened and closed such as Velcro™.

20. The apparatus of claim 19, wherein the hook and loop fastener comprises Velcro™
